(19) United States
(12) Patent Application Publication
(10) Pub. No.: US 2004/0253279 A1
(43) Pub. Date: Dec. 16, 2004

(54) Production of porous articles

(75) Inventors: Robert Terence Smith, Rotherham (GB); Rodney Martin Sambrook, Sheffield (GB)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(73) Assignee: Dytech Corporation Limited, Sheffield (GB)

(21) Appl. No.: 10/820,627

(22) Filed: Apr. 8, 2004

Related U.S. Application Data
(63) Continuation of application No. 09/269,999, filed on Jun. 28, 1999, now abandoned.
Application No. 09/269,999 is a 371 of international application No. PCT/GB97/02705, filed on Oct. 1, 1997.

(30) Foreign Application Priority Data
Oct. 4, 1996 (GB) 9620752.7

(51) Int. Cl. C04B 38/06; A61F 2/28; A61K 9/00

(52) U.S. Cl. 424/400; 501/81; 264/44; 264/624; 264/628; 623/16.11

(57) ABSTRACT

An aqueous dispersion of ceramic particles and containing a polymerisable monomer was foamed before polymerisation, e.g., using a catalyst and initiator, was started.

### CROSS-REFERENCE TO RELATED APPLICATIONS

This Application is a Continuation of U.S. application Ser. No. 09/269,999 filed Jun. 28, 1999, which is a U.S. National Stage Application under 35 U.S.C. §371, based on the International Application No. PCT/GB97/02705 filed Oct. 1, 1997.

The invention relates to the production of articles of controlled porosity.

In our European patent B-0598783 (Agent's ref: P00914EP) there is described and claimed a method of making a porous refractory article composed of refractory particles, the method comprising the steps of:

a) forming a dispersion comprising particles in a liquid carrier and containing a polymerisable monomeric material

b) introducing gas into the dispersion,

c) polymerising the foamed structure,

d) drying, to remove the liquid carrier to provide a solid article having pores derived from the bubbles; and

e) firing to remove the organic binder and to provide a ceramic bond.

Preferably the monomeric material is selected to polymerise by cross linking, whereby the liquid carrier and ceramic particles are held within the polymerised structure. In that disclosure there is mention that surfactants may be added to the dispersion to prevent premature settling of solids in the dispersion or to control the foaming characteristics. In Example IX Teepol surfactant is added to the dispersion. The foam was allowed to polymerise and ultimately sintered at 1450° C. for two hours.

The method of this prior patent is arranged to produce a variety of useful articles such as catalyst supports, flame supports; gas filters; air fresheners; ceramic armour; diesel particulate traps; insulation materials; artificial parts for the body; metal filters, reusable filters; liquid filters; storage and transportation for flammable and/or toxic materials, humidity sensors, chromatography; filter candles for filtration of hot combustion gases, diaphragms, membranes, refractory separators, phase dividers and electrolytes for high temperature fuel cells.

In our patent publication WO95/30631 (PCT/GB95/01063, Agents Ref P01204PCT) there is described and claimed a method of making a porous refractory article, the method comprising the steps of:

forming an aqueous dispersion of refractory particles including a polymerisable monomer component which on polymerisation generates an exotherm

adding a thermally activated gas generating substance

adding agents such as initiator and/or catalyst to cause the polymerisation

adjusting the temperature and/or pressure so that the gas generating substance generates the gas before or during the initial stage of polymerisation of the monomer component whereby the gas forms bubbles which under the heat of the exotherm interconnect to form an interconnected open pore structure.

By controlling the temperature and pressure the decomposition rate of the gas generating substance is controlled. This in turn provides a means of controlling the density of the end product. In this disclosure TRITON X-100, a surfactant was added as the last ingredient to the dispersion to stabilise the foam.

It is one object of the invention to provide a method of making a porous article having controlled levels of porosity, interconnectivity, pore size, and mechanical properties suitable for use in various applications.

In one aspect the invention provides a method of making a porous article comprised of bonded particles, the method comprising the steps of:

1) forming a dispersion comprising a liquid carrier and the particles and a polymerisable monomeric material;

2) adding a surfactant in a concentration according to the desired density of the porous article and then introducing bubbles of gas with agitation to form a foam and allowing or causing the bubbles to coalesce to form cells some of which form struts;

3) polymerising the monomeric material;

4) drying the structure to remove the liquid carrier and provide a solid article having pores derived from the bubbles; and

5) firing the article to provide a ceramic bond and to remove organic material present, whereby the porous article formed has a porosity of 20% to 95% and comprises cell walls and struts defining pores of pore sizes in the range of 15 to 150 micrometres.

The dispersion comprises particles having an average particle size less than about 5 micrometres and preferably 95% of the particles will be less than about 2 micrometres.

If the particles are larger than this size than the particles tend to settle or sediment. However, the particles can be much larger, say 100 micrometres or more, in which case agents will be present to control undesired settling; polymerisation of monomers is a suitable means of preventing settling. The particles will be chosen according to the intended end use. As will be explained later, for the preferred use hydroxyapatite is present either alone or with other particles. The other particles can include both oxides and non-oxides such as alumina, mullite, silicon carbide, silicon nitride, zirconia, titanium oxide and the like.

The content of the solids in the dispersion will tend to be about 10% by weight as a minimum and about 90% by weight as a maximum; a preferred range is from about 40% to about 80% by weight.

Conveniently the liquid carrier is water but it may be organic, e.g. alcohol, glycol or the like; or a mixture.

In order to produce a dispersion with the required solids loading and a suitable rheology, it is preferred to add dispersing agents to the suspension. The dispersing agents are commonly used throughout the ceramic industry and suitable agents in each case can be determined by the man skilled in the art. The colloidal stability may be controlled by the adjustment of the pH, in addition to or alternating to the dispersion agents.

Advantageously surfactants (which can be cationic, anionic or non-ionic) are present in the dispersion. In low viscosity systems where the ability of a system to foam is controlled by the surface tension, the surfactant type and concentration can have a direct influence not only on the level of porosity within the system but also the pore size and the interconnectivity. The concentration of surfactant can be used to influence the, bubble size with a foam and the rate of coalescence, both of which can be used to influence the final pore size.

By controlling the rheology of the suspension it is possible to influence the rate of coalescence of the foam structure. A slurry which exhibits a low degree of pseudoplasticity coupled with a low bulk viscosity is susceptible to foam collapse and rapid bubble growth. In contrast a slurry which exhibits a high degree of pseudoplasticity is subject to long term stability and a less rapid bubble growth although at an acceptable level. As the bulk viscosity of the system increases the controlling effect of the surfactant diminishes.

Another factor which influences the growth of the foam structure is the period before the onset of polymerisation. This period can be controlled by the addition levels of the initiator and catalyst. By controlling these levels as well as the oxygen concentration within the foaming gas the length of time before polymerisation starts can be controlled between an instantaneous polymerisation and one which starts after a period, which can be up to 20 minutes or more. Our evaluations suggest that this period has a major influence on cell structure where the porous article is to be used as a bone substitute.

In a further feature of the invention the process of increasing the cell size within a foam can be encouraged by the application of a partial vacuum to a foam before the commencement of polymerisation until after the end of polymerisation. At the end of polymerization the stable foam is brought back to atmospheric pressure. This additional step to the foaming process can be used to increase the rate at which the final desired large cell size can be achieved.

Other additives may be present, e.g. viscosity control agents, reinforcing fibres or particles, retarders for polymerisation; and the like.

The bubbles of gas may be introduced in any convenient way, although the preferred methods are listed in our previous patent. The gas used for foaming can be air, although an inert foaming gas such as nitrogen or argon is preferred. To produce a foam with a large pore structure and a high degree of porosity, one could either introduce bubbles of the correct size and quantity into the suspension or cause smaller bubbles to grow by a process of coalescence. The latter is preferred at the moment

In order to produce the small bubbles with the foam mechanical agitation is preferred.

Our investigations have shown that the structure of the foam can be broken into four distinctly different areas:

the cells or bubbles

the struts

the windows, and finally

the particulate microstructure

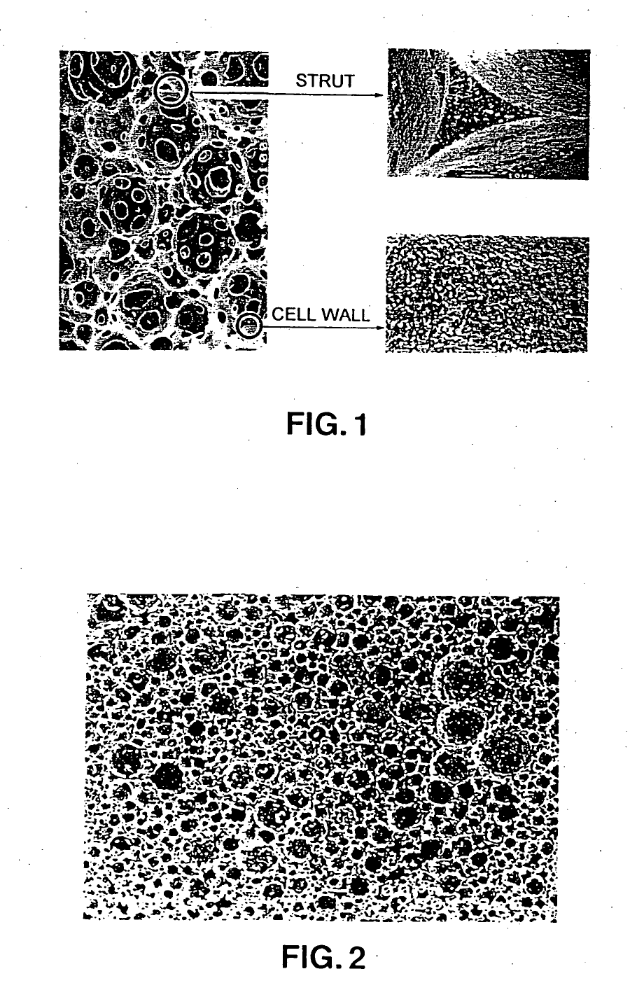

These structures can be seen in FIG. 1 of the accompanying drawings and are explained as follows.

A number of cells can be seen in the left hand photo in FIG. 1. These cells are formed as a result of introducing a gaseous phase into the ceramic suspension. Where these cells touch there is a tendency for the cell to change shape to maximise the packing density and for the slurry to drain from the point of contact towards the strut. A strut can be seen in the top right hand photo in FIG. 1. These struts are the building block for the foam structure. At the point of contact between each cell a thin film of liquid exists. During the later gelling and binder burnout steps this film is removed to leave what we call the cell “windows”. These are the connecting holes which interlink adjacent cells and can be clearly seen in the left hand photo in FIG. 1. The final structure is that which forms the cell walls and struts.

It is a feature of the invention that the final articles formed consist essentially of the starting ceramic materials only, so avoiding the need for the removal of residual secondary e.g. inorganic binders. The article can thus consist of ingredients acceptable for medical use, e.g. as bone grafts for orthopaedic, surgical, dental and like uses both for humans and animals. There will always be a need to replace bone lost as a consequence of traumatic or non-traumatic events. Bone substitute materials are available and approved for clinical use. These materials have been successfully used in orthopaedics, dentistry and facial plastic surgery. Among the types of bone graft materials used, particular interest has been shown in the porous types which can provide a scaffold for in growth of connective tissue and bone. Studies have shown that pore sizes less than 10 micrometre prevent ingrowth of cells, pore sizes of 15 to 50 micrometre encourages fibrovascular ingrowth; pore sizes of 50-150 micrometre result in osteoid formation; and pore sizes greater than 150 micrometre facilitate the ingrowth of mineralized bone. Different approaches have been taken to try and mimic the hydroxyapatite frame work within both the cortical and cancerous bone. One material is based on the conversion of a coralline structure to hydroxyapatite material. With this process the selection of the coral with the correct pore structure is imperative before conversion takes place. Two corals were eventually selected exhibiting two different pore structures. These two pore structures are intended to replicate the different structures in cortical and cancellous bone. It is a feature of this invention that synthetic articles made by the method may be used as bone graft materials of high acceptability.

Hydroxyapatite [Ca₁₀(PO₄), (OH)₂] is an ideal candidate starting material. This material belongs to a group of calcium phosphates which are being considered as bone substitute materials. The invention is applicable to hydroxyapatite and any other calcium phosphate (where the Ca/P atomic ratio may vary widely). In addition to this group of materials it may be advantageous to create an interconnected structure in another ceramic material such as alumna or zirconia for mechanical property reasons and either use the material as produced, or coated with a more bioactive material such as hydroxyapatite. It is another feature of this invention that the materials known as “Bioglass” could be converted in highly porous structures using this method.

The polymerisation preferably proceeds by crosslinking of reactive organic monomers. Examples include acrylates, such as ammonium acrylate or hydroxyethyl methacrylate; or the like. Preferably the monomers are dissolved in water or other liquid carrier to give a premix solution. To this solution are added an initiator and either heat or a catalyst are used to induce free radical vinyl polymerisation. In another variation, the dispersion includes a monosaccharide such as galactose, which can be condensed to form a dimer, trimer or polymer, to have the same effect.

The foamed composition may be allowed or caused to acquire sufficient green strength to allow it to be moved from the parent container or mould. The composition may be subjected to drying to drive off the liquid. In the case of water, drying can be carried out below about 100° C. in an oven or using high frequency drying equipment.

The pore sizes in the formed article can be controlled remarkable uniform to yield a material with a pre-determined pore size and level of interconnectivity. The true porosity may range from about 20% to about 95%. The article formed is relatively robust after polymerisation and strong enough to be machined after removal of the liquid carrier.

The firing temperature and duration are selected according to the nature of the particles, e.g. alumina generally requires a higher sintering temperature than hydroxyapatite.

In one more specific aspect the invention provides a method of making a porous article composed of bonded particles, the method comprising the steps of:

a) forming a dispersion comprising a liquid carrier and the particles and a polymerisable monomeric material;

b) forming a foam of the dispersion;

c) polymerising the foamed structure

d) drying the structure to remove the liquid carrier and provide a solid article having pores derived from the bubbles, and

e) firing the article to remove the organic binder and provide a ceramic bond.

characterised in that small bubbles of gas are introduced in the dispersion with agitation to form the foam and are allowed or caused to coalesce before the polymerisation, and in that the firing is carried out at a temperature appropriate to the growth of bone cells.

It is a feature of this invention to provide an article having a highly microporous structure if the sintering procedure is controlled. This microporous structure can have advantages in certain applications e.g. it may be infilled with certain drugs such as antibiotics or growth factors, to act as a slow release agent at the site of an implant and it appears to encourage the easy attachment of in-growing bone cells compared to a dense microstructure.

The formed article may be in a variety of shapes, e.g. in the form of granules, bars, cylinders or rods, blocks or the like.

In order that the invention may be well understood it will now be described by way of illustration only by reference to the following examples and micrographs:

### EXAMPLE I

Hydroxyapatite powder, ammonium acrylate monomer, methylenebisacrylamide, water, the ammonium salt of polyacrylate and the ammonium salt of polymethacrylate were mixed together to form a slurry which was subjected to a high shear mixer in order to remove any agglomerates within the slurry. This was transferred to a glove box within which the oxygen concentration was approx. 0.1%. A surfactant TERGITOL TMN10 was introduced into the slurry and the whole was agitated in a mixer designed to introduce air so that a foam will be formed. The amount by which the ceramic solid is foamed is dependant on the final density required, the solids content of the slurry and the shrinkage which will occur at the later stages of drying and firing. The amount of surfactant added determines the extent of foaming, and this was selected to achieve the required final density. Once the foam density was achieved, ammonium persulphate, (initiator) and tetramethylethylenediamine (catalyst) were injected into the foam to cause the acrylate monomer to start to polymerise. The time before the onset of polymerisation was about 1.5 minutes.

The mixture was restirred and allowed to stand. Polymerisation began after about 1.5 minutes. A photo of the microstructure produced after an idle time of 1.5 minutes is shown in FIG. 2. Once polymerised the foam was removed from the mould and allowed to dry at room temperature for 2 days before being forced dried at 60° C. in an oven.

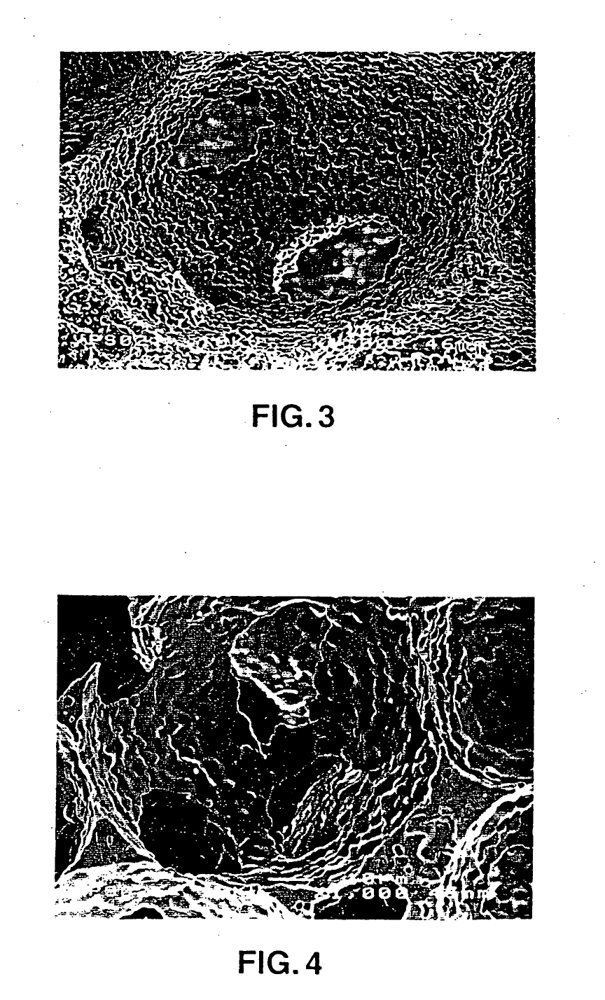

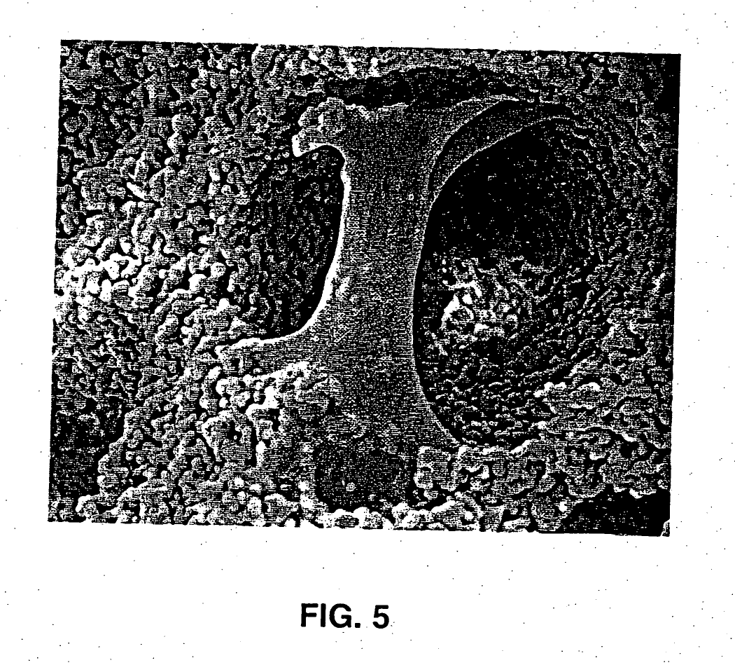

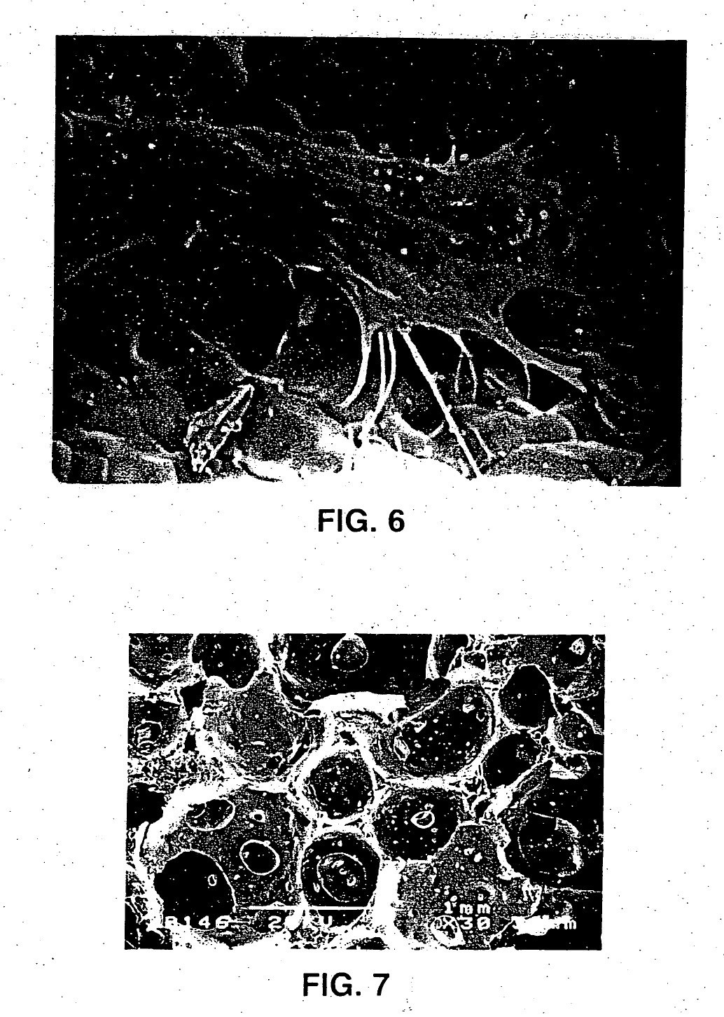

At this point the “green” ceramic can easily be machined into the desired shape. The “green” article was heated in a furnace to remove the organic binder and to cause the ceramic microstructure to densify. The sample was split in two and fired at two different temperatures. Sample 1 shown in FIG. 3 and sample 2 in FIG. 4 were fired at 1250° C. for 2 hrs and 1350° C. for 2 hrs respectively. It can be seen that the degree of microstructural densification can be adjusted with the sintering conditions. Sample 1 exhibits a highly connected microstructure whereas the microstructural porosity has been removed in sample 2. Live human bone cells were cultured. Both samples 1 and 2 were immersed in the cultures and FIGS. 5 and 6 show the results after 36 hrs immersion for sample 1 and 2 respectively. The bone cells can be clearly seen on the surface of the cell walls. From these Figures it appears easier for the bone to grow within the undersintered microstructure than the fully densified structure.

### EXAMPLE II

The method of Example I was repeated except that the rate of addition of the initiator and the catalyst were selected so that the time before onset of polymerisation was 16 minutes instead of 1.5 minutes. A highly porous foam exhibiting a larger cell size as shown in FIG. 7 resulted. It can be seen from the different Figures that the time before the onset of polymerisation has had a major influence on the cell structure.

The Figures of the accompanying drawings are microphotographs as follows:

FIG. 1 is a general foam;

FIG. 2 is a foam produced in Example I taken after an idle time of 1.5 minutes;

FIG. 3 is the polymerised foam of Example I fired at 1250° C. for 2 hours;

FIG. 4 is the polymerised foam of Example I fired at 1350° C. for 2 hours;

FIG. 5 is the fired product shown in FIG. 3 after being immersed in a bone cell culture for 36 hours;

FIG. 6 is the fired product shown in FIG. 4 after being immersed in a bone cell culture for 36 hours; and

FIG. 7 is the foam produced in Example II.

1-19. cancelled.

20. The method according to claim 21, including adding a drug to the pores of the article.

21. A method of making a porous article composed of bonded particles and having a predetermined level of porosity, pore size and interconnectivity, the method consisting essentially of:
a) forming a dispersion comprising a liquid carrier, particles to be bonded and a polymerizable monomeric material;
b) adding a surfactant and then introducing small bubbles of oxygen containing gas into the dispersion with agitation to form a foam which is allowed or caused to coalesce;
c) polymerizing the foamed structure;
d) adjusting the period from the formation of the foam to the start of the polymerization by adding initiator and catalyst therefor at rates selected to influence the structure of the pores to be present in the porous article;
e) drying the structure to remove the liquid carrier and provide a solid article having pores derived from the bubbles; and
firing the article to a temperature to remove the organic material and to undersinter the formed article and thereby form the porous article which has a porosity of 20% to 95% and comprises pore walls and struts defining pores of pore sizes in the range of 15 to 150 micrometers and in which bone cells may easily be attached.

22. The method according to claim 21, wherein the period until onset of polymerization is between an instantaneous polymerization and about 20 minutes.

23. The method according to claim 21, including the step of controlling the onset of polymerization by adjusting addition levels of an initiator and catalyst for polymerization of the monomeric material.

24. The method according to claim 21, wherein the particles in the dispersion are less than about 5 micrometers.

25. The method according to claim 21, wherein the particles are hydroxyapatite, oxides and non-oxides.

26. The method according to claim 21, wherein the content of the solids in the dispersion is about 10% to about 90% by weight.

27. The method according to claim 26, wherein the content of the solids is about 40% to about 80% by weight.

28. The method according to claim 21, wherein the liquid carrier is water, organic liquid or a mixture thereof.

29. The method according to claim 21, including the step of adding a dispersing agent to the dispersion.

30. The method according to claim 21, wherein the solid article is substantially dried and then fired at about 1250□ C for two hours.

31. The method according to claim 21, wherein the solid article is dried and then fired at about 1350□ C for about two hours.

32. A method according to claim 21, wherein the formed body has a true porosity of from about 20% to about 95%.

33. A method according to claim 21, wherein the formed body has pores in the pore size range of about 5 micrometers to about 20 micrometers.

34. A method according to claim 21, wherein the formed body has pores in the pore size range of about 50 micrometers to about 150 micrometers.

35. A method according to claim 21, wherein the formed body has pores having a pore size greater than about 150 micrometers.

36. A method according to claim 21, including a subsequent step of growing bone cells in the porous product.

37. A method according to claim 21, including a subsequent step of infilling the pores of the porous product with a drug.
